SEQUENCE LISTING (2 sequences)

SEQ ID NO: 1 - DNA, 20 bp, Artificial
  oligonucleotide
actaactactgtatcctgca

SEQ ID NO: 2 - DNA, 20 bp, Artificial
  oligonucleotide
tgctggtttctgttgtttgt

(19) United States
(12) Patent Application Publication
(10) Pub. No.: US 2016/0209405 A1
(43) Pub. Date: Jul. 21, 2016

(54) MAGNETIC DETECTION OF MERCURIC ION USING GIANT MAGNETORESISTIVE BASED BIOSENSING SYSTEM

(71) Applicant: Regents of the University of Minnesota, Minneapolis, MN (US)

(72) Inventors: Jian-Ping Wang, Shoreview, MN (US); Wei Wang, Minneapolis, MN (US); Yi Wang, Minneapolis, MN (US); Todd Klein, Wayzata, MN (US)

(21) Appl. No.: 14/676,620

(22) Filed: Apr. 1, 2015

Related U.S. Application Data
(60) Provisional application No. 61/974,276, filed on Apr. 2, 2014.

(51) Int. Cl. G01N 33/543; G01N 33/53; G01R 33/09

(52) U.S. Cl. CPC G01N 33/54326; G01R 33/093; G01N 33/5308

(57) ABSTRACT

A system may include a magnetic sensor including a free magnetic layer and a fixed magnetic layer; a sample container disposed over the magnetic stack; a plurality of capture DNA oligomers on a surface of the magnetic biosensor within the volume defined by the sample container above the magnetic stack, wherein each of the plurality of capture DNA oligomers includes at least one thymine base configured to bind to an Hg²⁺ ion; a magnetic field generator configured to generate a magnetic field that influences the free layer; and circuitry configured to measure a resistance of the magnetic sensor.

This application claims the benefit of U.S. Provisional Patent Application No. 61/974,276, filed Apr. 2, 2014, titled “MAGNETIC DETECTION OF MERCURIC ION USING GIANT MAGNETORESISTIVE BASED BIOSENSING SYSTEM.” The entire content of U.S. Provisional Patent Application No. 61/974,276 is incorporated herein by reference.

### TECHNICAL FIELD

The disclosure relates to sensors utilizing giant magnetoresistance.

### BACKGROUND

Contamination with mercury is an important environmental and health concern throughout the world. Mercuric ion (Hg²⁺) is stable and soluble in aquatic systems. Exposure to high amounts of mercuric ion may result in acrodynia (Pink disease) and damage to the nervous system and the kidneys. Furthermore, mercuric ion can be transformed to methyl mercury by microbial biomethylation. Methyl mercury can accumulate in bodies throughout the food chain, and is known to cause brain damage and other chronic diseases, including paralysis and death. Therefore, sensitive methods for the detection of Hg²⁺ in environmental monitoring are desired.

### SUMMARY

In general, the disclosure describes techniques and systems for detecting mercuric ion using giant magnetoresistive (GMR) biosensors and DNA chemistry. A GMR biosensor utilizing thymine-thymine pairs may be highly selective for Hg²⁺ ions and may possess high sensitivity and substantially real-time signal generation, allowing substantially real-time detecting of Hg²⁺ ion concentration in a sample. The systems described herein may have a detection limit of about 10 nanomolar (nM) or less Hg²⁺ ions in both buffer solution and natural water. 10 nM Hg²⁺ is the maximum recommended mercury level in drinking water regulated by U.S. Environmental Protection Agency (EPA). The magnitude of the dynamic range for Hg²⁺ detection may be as great as three orders of magnitude or more (e.g., about 10 nM Hg²⁺ to about 10 μM Hg²⁺). A GMR biosensor as described herein could be utilized for environmental monitoring, food safety testing, or both.

In some examples, the disclosure describes a system including a magnetic sensor comprising a free layer and a fixed layer; a sample container disposed over the magnetic stack; a plurality of capture DNA oligomers on a surface of the magnetic biosensor within the volume defined by the sample container above the magnetic stack, wherein each of the plurality of capture DNA oligomers includes at least one thymine base configured to bind to an Hg²⁺ ion; a magnetic field generator configured to generate a magnetic field that influences the free layer; and circuitry configured to measure a resistance of the magnetic sensor.

In some examples, the disclosure describes a magnetic biosensor array comprising: a plurality of electrical contacts located along at least one peripheral edge of the magnetic biosensor array; a sample container; a plurality of the magnetic biosensors each located adjacent to a surface of the magnetic biosensor array and comprising a magnetic sensor comprising a free layer and a fixed layer; and a plurality of capture DNA oligomers on a surface of the magnetic biosensor within the volume defined by the sample container above the plurality of the magnetic biosensors, wherein each of the plurality of capture DNA oligomers includes at least one thymine base configured to bind to an Hg²⁺ ion

In some examples, the disclosure describes a kit comprising a magnetic biosensor comprising a magnetic sensor comprising a free layer and a fixed layer; a sample container disposed over the magnetic stack; and a plurality of capture DNA oligomers on a surface of the magnetic biosensor within the volume defined by the sample container above the magnetic stack. The kit also may include a solution including a plurality of biotin-labeled DNA; a solution including a plurality of streptavidin labeled magnetic nanoparticles; and instructions for introducing a sample into the sample container, introducing the solution including the plurality of biotin-labeled DNA into the sample container, and introducing the solution including the plurality of streptavidin labeled magnetic nanoparticles into the sample container to detect a concentration of Hg²⁺ ions in the sample.

In some examples, the disclosure describes a method for forming a magnetic biosensor, the method comprising forming a magnetic sensor comprising a free layer and a fixed layer, wherein at least one of the free layer or the fixed layer has a magnetic moment oriented out of a major plane of the free layer or the fixed layer, respectively, in an absence of an external magnetic field; placing a sample container over the magnetic stack; and attaching a plurality of capture DNA oligomers to a surface of the magnetic sensor, wherein each of the plurality of capture DNA oligomers includes at least one thymine base configured to bind to an Hg²⁺ ion.

In some examples, the disclosure describes a method for detecting a concentration of Hg²⁺ ions in a sample, the method comprising introducing a sample including Hg²⁺ ions into a sample container, wherein the sample container defines a volume adjacent to a magnetic biosensor comprising a magnetic sensor comprising a free layer and a fixed layer, a sample container disposed over the magnetic stack, and a plurality of capture DNA oligomers on a surface of the magnetic biosensor within the volume defined by the sample container above the magnetic stack; introducing a solution including a plurality of biotin-labeled DNA into the sample container; introducing a solution including a plurality of streptavidin labeled magnetic nanoparticles into the sample container; and detecting a resistance of the magnetic sensor.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram illustrating an example detection process for Hg²⁺ ions.

FIG. 2 is a conceptual diagram illustrating an example magnetic chip including a plurality of GMR sensors.

FIG. 3 is a diagram illustrating an example transfer curve of a GMR sensor, including the resistance change of the sensor versus applied external magnetic field change along a minor axis of the GMR sensor.

FIGS. 4(a)-4(f) are a series of conceptual diagrams illustrating an example technique for forming a GMR sensor.

FIG. 5(a) is an optical micrograph illustrating a shape of an example sensor.

FIG. 5(b) is a conceptual diagram illustrating detailed size of an example GMR sensor.

FIG. 6 is photograph illustrating an entire, example GMR sensor-based detection system.

FIG. 7(a) is an example image illustrating a sciFLEXARRAYER S5 system (Scienion, Germany) used to deposit (print) capture DNA oligomer.

FIG. 7(b) is an example image of a GMR sensor array without printed samples.

FIG. 7(c) is an example image of a GMR sensor array with printed DNA solution.

FIG. 8 includes a series of fluorescence microscopy images of capture DNA immobilized on surfaces and a bar graph illustrating fluorescence density versus concentration of capture DNA.

FIG. 9 includes a series of fluorescence microscopy images of biotin-DNA bound to sensor surfaces and a bar graph illustrating fluorescence density versus concentration of biotin-DNA.

FIG. 10 is an example graph of fluorescence density versus concentration of Hg²⁺.

FIG. 11(a) is an image illustrating an example 4-inch silicon wafer, which, in some examples, may produce 21 full GMR biochips and 4 fragmentary GMR biochips.

FIG. 11(b) is an image illustrating the size of an example GMR biochip compare to a U.S. quarter.

FIG. 11(c) is an example plot of binding signal versus time for example Hg²⁺ assays of various concentrations

FIG. 11(d) is an example bar diagram illustrating average signals (with standard deviation) for mercuric ions (Hg²⁺) of different concentrations in buffer.

FIG. 12 illustrates a series of scanning electron microscopy (SEM) images of surfaces after being exposed to mercuric ions of different concentrations in buffer.

FIG. 13 is a plot of change in signal strength versus time for a sensor after being exposed to mercuric ions of different concentrations in buffer.

FIG. 14 illustrates example SEM images of MNPs binding on a pregnancy-associated plasma protein A biotinylated antibody modified GMR sensor under different magnifications.

FIG. 15 is a diagram illustrating example GMR sensor signal versus the number of bound MNPs per μm² on a GMR sensor surface.

FIG. 16 is a bar diagram illustrating the change in signal of a GMR biosensor for each of six metals.

FIG. 17 is a bar diagram illustrating average signals for various Hg²⁺ concentrations in natural water.

FIGS. 18A-18D are conceptual diagrams that illustrate an example technique by which a magnetic biosensor may detect a concentration of an analyte in a sample.

FIGS. 19A and 19B are conceptual diagrams that illustrate another example technique by which a magnetic biosensor may detect a concentration of an analyte in a sample.

### DETAILED DESCRIPTION

Traditional methods to detect mercury include atomic absorption spectrometry, cold vapor atomic fluorescence spectrometry, and inductively coupled plasma mass spectrometry. However, these tests may be expensive, non-portable, and rely on central laboratories to perform the tests. Recently, several methods have been developed for detecting Hg²⁺ using an electrochemical sensor, a triboelectric sensor, surface plasmon resonance, a quartz crystal microbalance, and quantum dots, etc. Besides these techniques, one notable and fast-developing approach is using colloidal gold nanoparticles which have been widely used in biomedical areas. Gold nanoparticles are advantageous for Hg²⁺ detection with high sensitivity and high selectivity, and are feasible for in-field analysis while combining with small molecules, proteins, and DNA.

Giant magnetoresistive (GMR) sensors have been widely and successfully used in hard drive heads since the late 1990s. As described herein, a GMR sensor may be used as a biosensor. GMR biosensor technology has the merits of relatively low cost, relatively high sensitivity, and substantially real-time signal read-out. The fabrication and integration of the GMR biosensors are compatible with the current Very-Large-Scale Integration (VLSI) and System on Chip (SOC) technologies, so it has great potential for eventually realizing point of care and portability with low cost. Furthermore, one of the fundamental advantages of a GMR biosensor is that the magnetic background of biological and environmental fluids is usually negligible. In contrast to colorimetric methods that require the use of light, there is no worry of magnetic signal being interfered by the sample matrix (e.g., water).

In accordance with examples of this disclosure, the final output signal for GMR biosensing originates from the stray magnetic field introduced by bound superparamgnetic magnetic nanoparticles (MNPs) at the GMR sensor surface. The bound MNPs are magnetized as magnetic dipoles by an applied alternating magnetic field. The magnetic dipoles generate the magnetic field that is sensed by the GMR sensor. A greater number of bound MNPs generally leads to a higher detection signal. Therefore, to detect Hg²⁺ ions, the biosensor should be designed such that the number of bound MNPs is dependent on the number of Hg²⁺ ions in the sample.

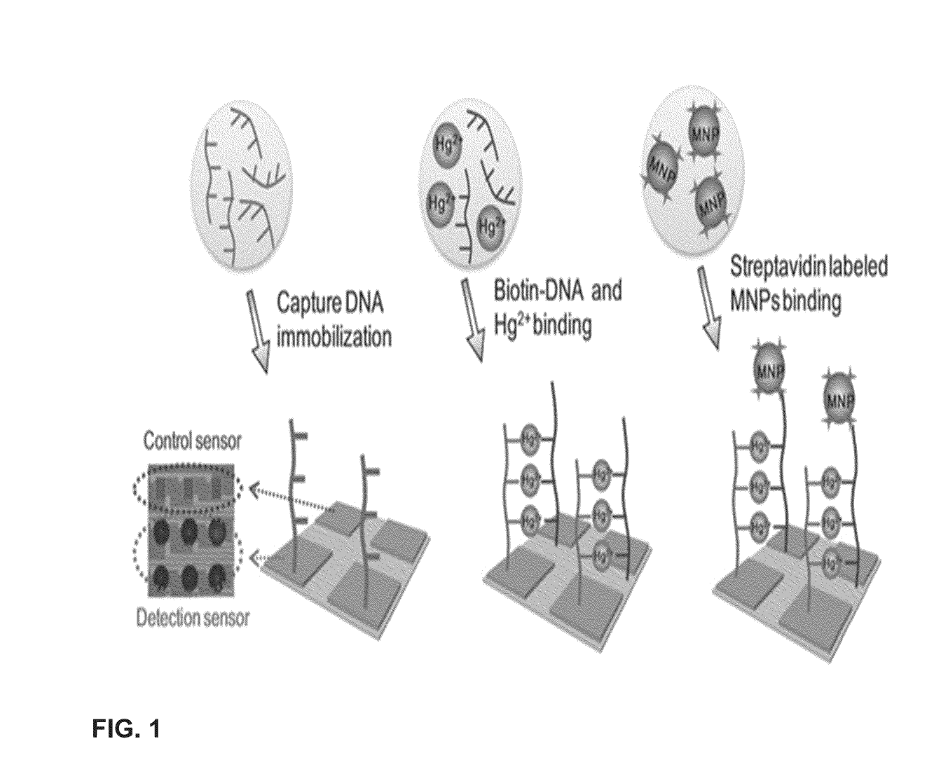

Hg²⁺ ions can specifically bind between two DNA thymine bases to form a thymine-Hg²⁺-thymine (T-Hg²⁺-T) pair. The Hg²⁺ mediated T-T base pair may be at least as stable as normal Watson-Crick base pairs. The magnetic biosensor described herein utilizes this T-Hg²⁺-T complex chemistry. Complementary DNA with deliberately designed T-T mismatches is introduced and combined with a GMR biosensing system for sensitive and selective Hg²⁺ detection. The detection process is briefly illustrated in FIG. 1. FIG. 1 is a conceptual diagram illustrating an example detection process for Hg²⁺ ions. This detection architecture is similar to a sandwich DNA hybridization assay, but the target DNA is replaced by Hg²⁺ ions.

As shown in FIG. 1, after capture DNA oligomers are immobilized on the GMR sensor surface, biotin labeled DNA (biotin-DNA) with T-T mismatches relative to the capture DNA are added with the sample, which may include Hg²⁺ ion. In the absence of Hg²⁺, biotin-DNA is rarely hybridized to immobilized capture DNA because of the mismatched base pairs. In contrast, the biotin-DNA can be bound and hybridized to the capture DNA oligomers attached to the GMR sensor surface in the presence of Hg²⁺ due to the T-Hg²⁺-T complex and Watson-Crick base pairing. The amount of bound biotin-DNA increases as the amount of Hg²⁺ increases. Once the biotin-DNA has bound to the capture DNA oligomers via the T-Hg²⁺-T complex and has reached an equilibrium state, the sample may be removed and the sensor rinsed to remove unbound biotin-DNA and other constituents of the sample. Streptavidin labeled MNPs then may be introduced to the sensor, and may bind with the bound biotin-DNA. In this way, an increased amount of Hg²⁺ in the sample may lead to an increased number of MNPs bonded to the biotin labeled DNA.

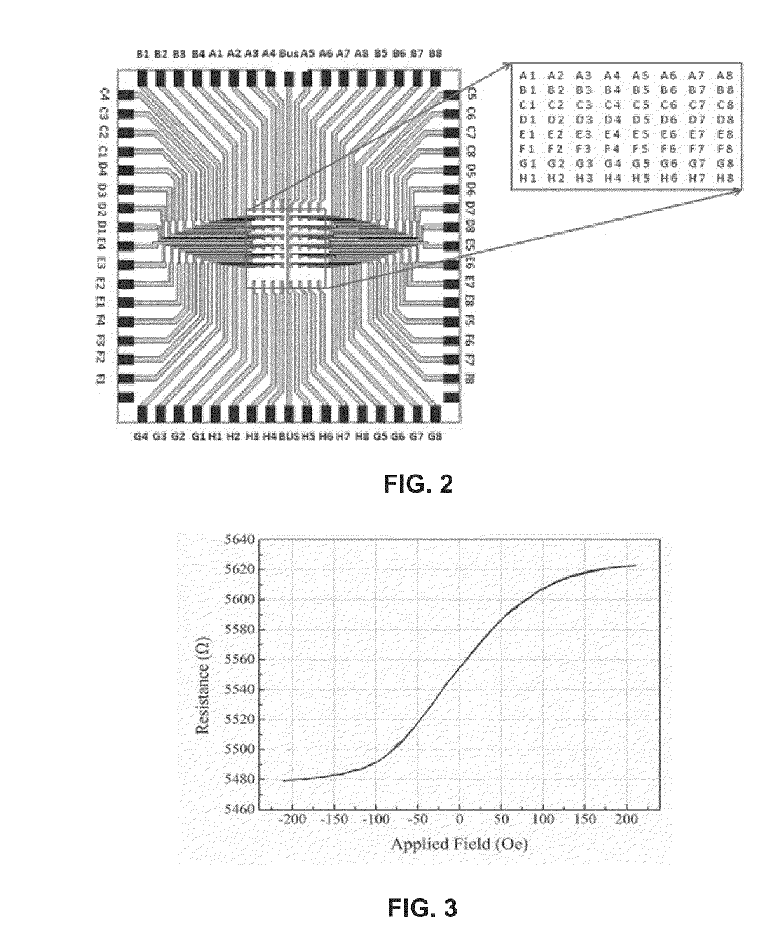

FIG. 2 is a conceptual diagram illustrating an example magnetic chip including a plurality of GMR sensors. The chip illustrated in the example of FIG. 2 included 64 GMR sensors and was fabricated using a photolithography technique. The layout and size for the chip and sensor are shown in FIG. 2. The 64 sensors were symmetrically arranged in an 8×8 array, and this would be convenient for automatic spotting with biomolecules in sensor surface functionalization. The sizes of one GMR chip and one sensor are about 16×16 mm and 120×175 μm, respectively. Each sensor had been numbered and accordingly connected to peripheral contact pads on the periphery of the chip via contact lines. These numbered pads serve as one electrode for each sensor, and the two bus pads connected to all sensors serve as another electrode. Even if one bus pad breaks, all sensors can still work via another bus pad. The two unnumbered pads are used as two reference points for automatic spotting rather than used for electronic purposes. Unlike microscope slide- or micro-titer plate-based diagnostic techniques, capture biomolecules should be accurately spotted and immobilized on sensor surface for GMR biosensor. In some examples, 64 sensors on one magnetic chip may be appropriate taking into account sensor spacing and layout of connecting wires. However, this is not the limit of sensors on one GMR chip, and GMR sensors could be scaled to over 100,000 sensors per cm².

In some examples, the GMR sensor including a pinned magnetic layer, whose magnetic orientation does not change under an applied magnetic field of the strength utilized in the sensor, and a free magnetic layer, whose magnetic orientation may change when exposed to a magnetic field, such as the stray magnetic field generated by MNPs. In some examples, the magnetic orientation of the pinned layer may be aligned to a minor axis of the GMR sensor. A transfer curve of the GMR sensor may be generated by measuring the resistance change of the GMR sensor as the applied external magnetic field is changed by sweeping the field strength along the minor axis of the GMR sensor. As shown in FIG. 3, an example GMR sensor may have a maximum resistance of 5623Ω in the antiparallel state and a minimum resistance of 5479Ω in the parallel state, giving a magnetoresistance ratio (MR) of about 2.6%. The magnetic orientation of free magnetic layer may be along the major axis because of its long strip shape as no external field was applied during annealing. The transfer curve has a linear part in the range of −50 Oe to 50 Oe, which is desired for GMR bio-sensing.

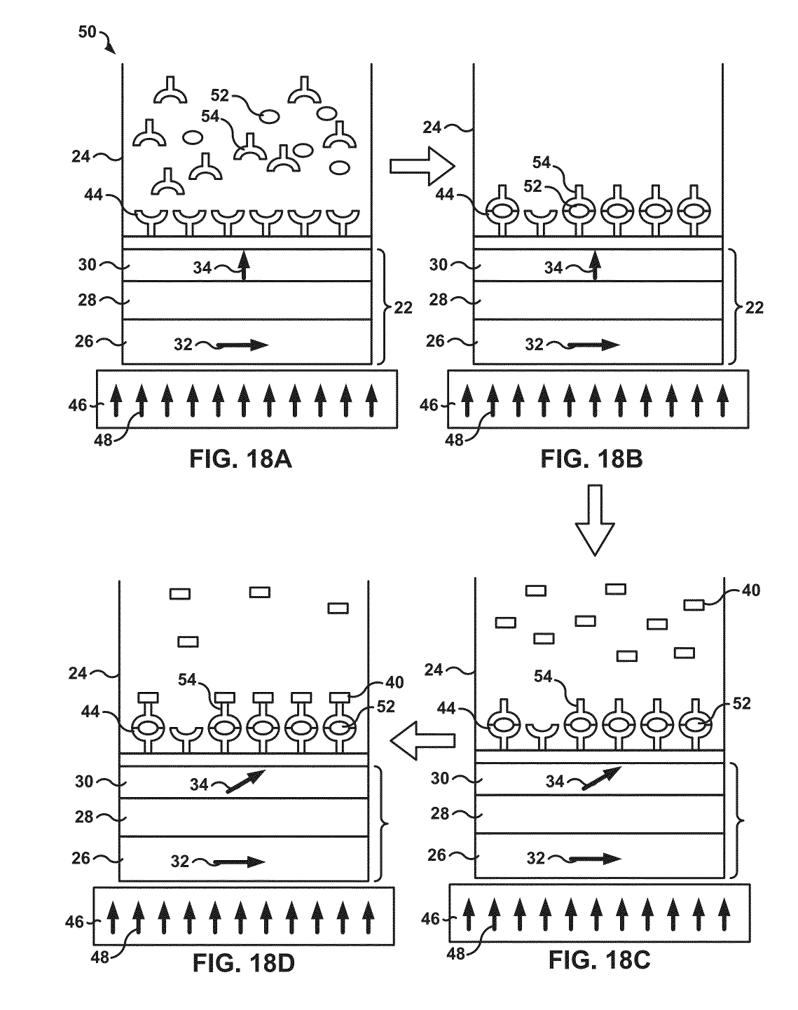

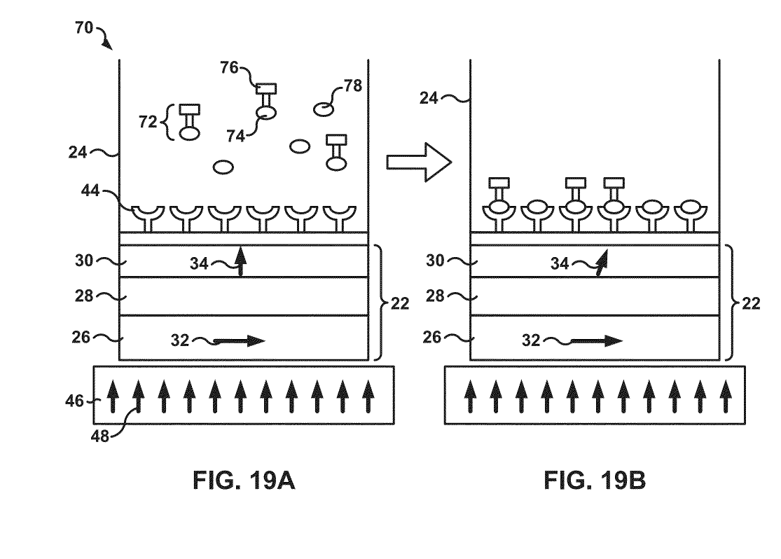

In this way, in some examples, the stable magnetic orientations of the free magnetic layer and the pinned magnetic layer may be in the plane of the GMR sensor, and may be substantially perpendicular. In other examples, the stable magnetic orientations of the free magnetic layer and the pinned magnetic layer may be in the plane of the GMR sensor and may be substantially parallel, substantially antiparallel, or at another non-parallel and non-perpendicular angle. In still other examples, such as those illustrated below in FIGS. 18(a)-18(d), 19(a), and 19(b), the stable magnetic orientation of one or both of the free magnetic layer and the pinned magnetic layer may be canted out of the plane of the GMR sensor, e.g., may be substantially perpendicular to the plane of the GMR sensor.

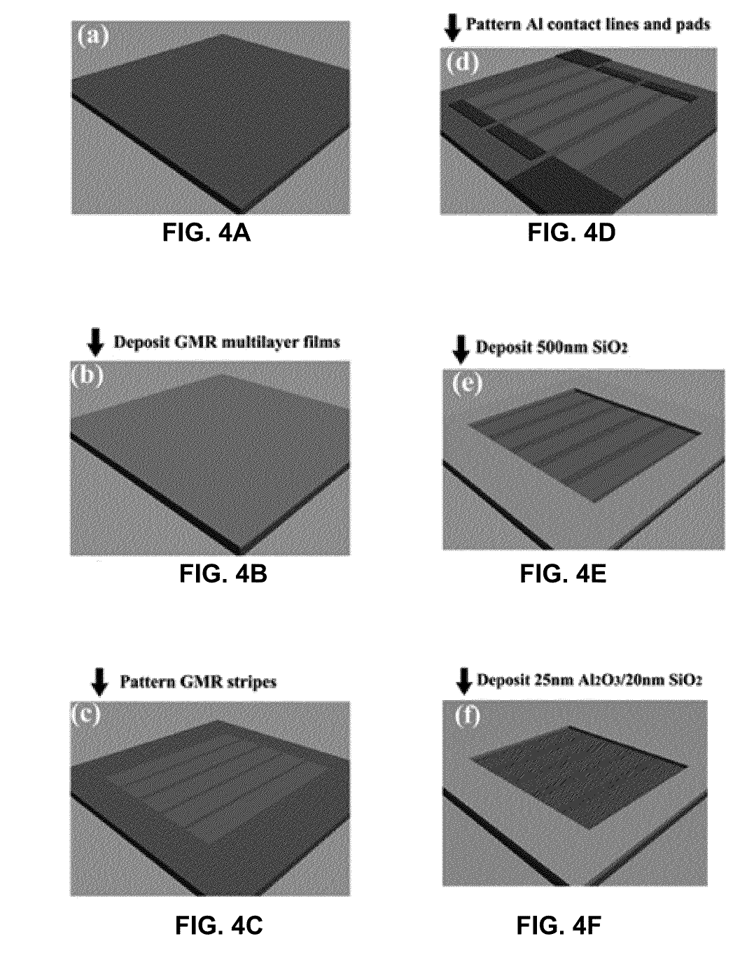

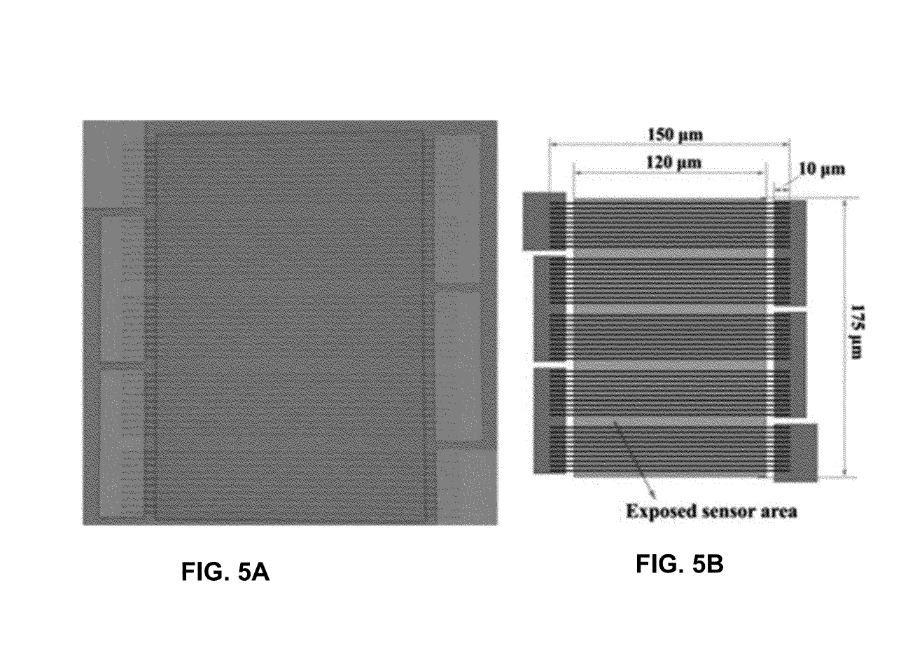

FIGS. 4(a)-4(f) are a series of conceptual diagrams illustrating an example technique for forming a GMR sensor. As shown in FIGS. 4(a) and 4(b), first, GMR multilayer films are deposited on a substrate. In some examples, the multilayer films may include Ta, IrMn, CoFe, Cu, CoFe, NiFe, and Ta, from the bottom layer up. The GMR stripes are then patterned, as shown in FIG. 4(c). In this example, the pattern includes five groups often strips each. The conductive contact lines and pads are then formed, as shown in FIG. 4(d). The shape of the sensor was visualized and confirmed under optical microscope, as shown in FIG. 5(a). FIG. 5(b) illustrates detailed size of an example GMR sensor. The example GMR sensor includes 50 stripes in 5 stripe groups of 10 stripes each connected in parallel. The dimension of one stripe is about 150 μm by about 750 nm. The width of stripes was confirmed using a JOEL 6500 scanning electron microscope (SEM). The GMR chip surface was coated with 500 nm thick SiO₂ except exposed sensor area and contact pads on the periphery of the chip, as shown in FIG. 4(e). The active length of one stripe is 120 μm, and the gap between stripes is about 2 μm. The exposed sensor area is about 120 μm by about 175 μm. Finally, in some examples, a protective bi-layer including Al₂O₃ and a top layer SiO₂ may be formed on the Ta layer, as shown in FIG. 4(f). In some examples, the designed and fabricated GMR biochip in this work includes 64 GMR sensors. Each GMR sensor may operate independently. A single 4-inch silicon wafer with a GMR multilayer stack can produce 21 full GMR biochips. The fabrication cost could be dramatically reduced if a mass production process with a larger wafer (e.g. 12 inch), a smaller chip size, or both is employed. In some examples, a plastic substrate or a polymer substrate may be used for the fabrication of magnetic chips with low cost. For this purpose, soft-lithography processes (e.g., stamping the chemicals on the substrate) may be used for patterning the magnetic chips.

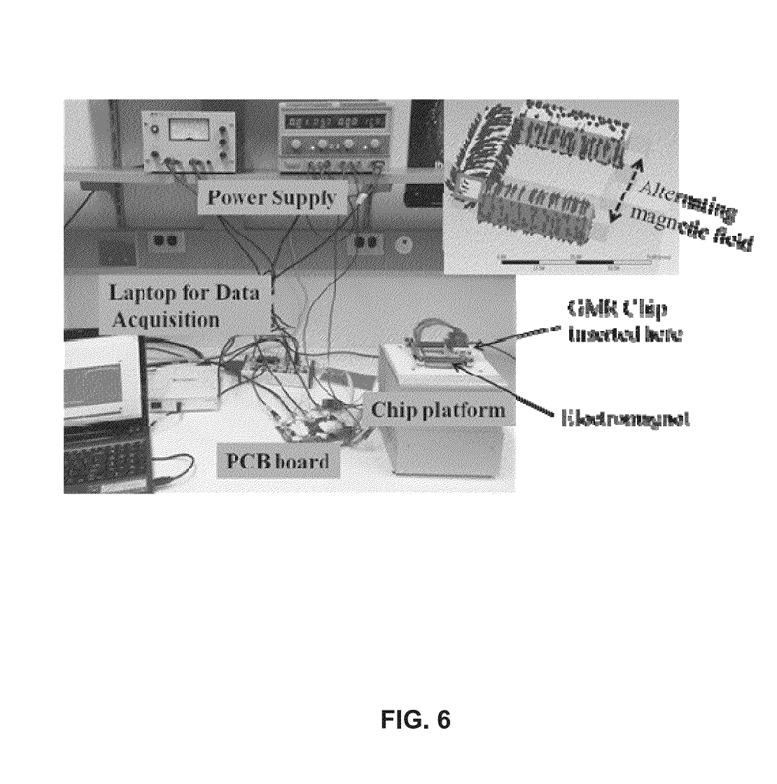

An photograph of example of an entire GMR sensor-based detection system is shown in FIG. 6. The designed chip holder can be fixed on a connection stage which connects the GMR sensor(s) with a printed circuit board (PCB). In this way, the system is inexpensive and can be reused thousands of times. The system includes a power supply, a Wheatstone bridge PCB, a laptop, and a chip platform with an electromagnet. The electromagnet has a soft iron core and is wound with copper wires. A schematic illustration of the electromagnet is shown at the top-right corner of FIG. 6. While an alternating current is applied to the coil, an alternating magnetic field is produced. This alternating field will magnetize the bound MNPs on the sensor surface during the signal measurement.

GMR spin valve films were deposited at the University of Minnesota using a Shamrock Magnetron Sputter System onto Si/SiO₂ (1000 Å) substrate. The multi-layer films were top-down composed of Ta (50 Å)/NiFe (20 Å)/CoFe (10 Å)/Cu (33 Å)/CoFe (25 Å)/IrMn (80 Å) Ta (25 Å). An anti-ferromagnetic IrMn layer was used to pin the fixed magnetic CoFe layer, and the free layer consisted of CoFe and NiFe bi-layers. A GMR chip including 64 GMR sensors in an 8×8 array was fabricated with photolithography techniques, as described above with respect to FIG. 2. Protective bi-layers of 25 nm Al₂O₃ and 20 nm SiO₂ were coated on chip surface by ALD (Atomic Layer Deposition) and PECVD (Plasma Enhanced Chemical Vapor Deposition), respectively. The bi-layer was used to prevent leakage current and surface SiO₂ was convenient for further surface functionalization. The resulting GMR chip was similar to that shown in FIG. 4. The GMR chip was annealed at 200° C. for 1 h under 4.5 kOe magnetic field and the field orientation was along minor axis of GMR sensor. The magnetic orientation of the pinned layer could be fixed along the minor axis after annealing treatment.

The GMR chip surface was functionalized using 3-aminopropyltriethoxy silane (APTES) and glutaraldehyde (Glu). After thoroughly washing the SiO₂ surface layer with acetone, methanol, and isopropanol, the chip was dried using nitrogen gas. The GMR chip was dipped in 0.5% APTES solution (in toluene) for 15 min, then washed with acetone and deionized (DI) water. The APTES-modified chip was placed in 5.0% Glu solution (in PBS buffer, 1×, pH 7.4) and incubated for 5 h, followed by washing with DI water and drying with nitrogen gas. After APTES-Glu modification, aldehyde groups were attached onto the sensor surface, so biomolecules containing amino groups, such as proteins and amine labeled DNA can be immobilized on GMR sensor surface.

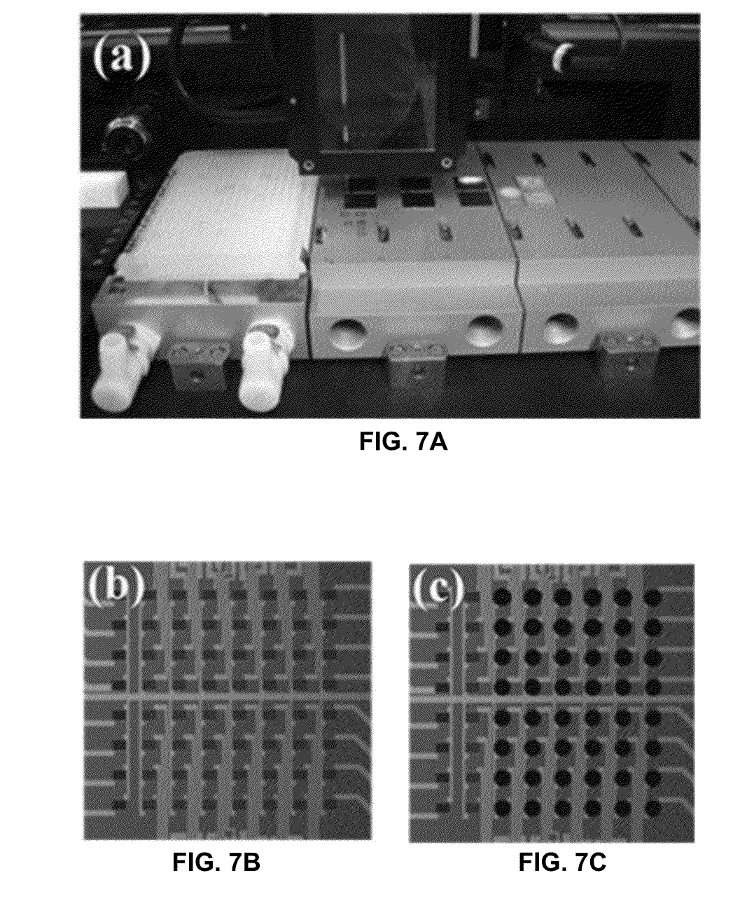

The capture DNA oligomer (5′/ACTAACTACTGTATCCTGCA/3AmMC6T/3′) with amino modification at the 3′ end was purchased from Integrated DNA Technologies, Inc (20 nmol/mL in PBS buffer, 1×, pH 7.4). The capture DNA oligomer was spotted on individual GMR sensors, and some of the sensors within a chip were not spotted, to be used as control sensors, as shown in FIG. 7(c). FIG. 7(a) is an image illustrating a sciFLEXARRAYER S5 system (Scienion, Germany) used to deposit (print) the capture DNA oligomer. FIG. 7(b) is an image of the GMR sensor array without printed samples. FIG. 7(c) is an image of a GMR sensor array with printed DNA solution. The 16 sensors in the left two columns were used as control sensors and left unprinted. The distance (pitch) between the centers of adjacent spots is about 400 μm.

The printed GMR chip was incubated for about 24 hours at room temperature under a relative humidity of about 90%. After being rigorously rinsed with 0.2% SDS (sodium dodecyl sulfate) solution three times to remove unbound capture DNA oligomers, the printed GMR chip was further washed with ultrapure water. For inactivating surplus aldehyde groups and reducing non-specific binding, 20 μL NaBH₄ solution (dissolving about 1.0 mg NaBH₄ in about 400 ILL PBS (1×) and 100 μL ethanol) was added the GMR chip surface and incubated for approximately 5 min. After three washes with ultrapure water, the GMR chip was immersed in hot water for several minutes to denature any annealed DNA. Then the GMR chip was rinsed thoroughly with ultrapure water and dried by nitrogen gas.

A bottomless reaction well made of polymethyl methacrylate (PMMA) was attached onto GMR chip surface. In some examples, the reaction well can allow a maximal liquid volume of 100 μL on a single sensor array area. A mixture solution was made of 50 nmol/mL Biotinylated DNA oligomer (5′ITGCTGGTTTCTGTTGTTTGT/BiotinBB-/3′, purchased from TriLink Biotechnologies), 0.01% polysorbate (polyoxyethylene (20) sorbitan monolaurate; Tween 20), 10 mM HEPES buffer (4-(2-hydroxyethyl)-1-piperazineethanesulfonic acid; pH=7.5), 100 mM NaClO₄, and Hg²⁺ with a predetermined concentration (0 nM, 10 nM, 100 nM, 1 μM, and 10 μM for the different samples). The Hg²⁺ solution was prepared by diluting a concentrated stock solution (1 mM determined by cold vapor atomic fluorescence spectrometry). The mixture solution (100 μL) was loaded into reaction wells and incubated for about 2 hours at about 40° C. After that, the GMR chip was washed with 0.2% SDS at room temperature for 5 minutes, and rinsed with ultrapure water three times, followed by being dried by nitrogen gas. The GMR chip was tightly sealed and kept in a refrigerator at about 4° C. before its signal measurement.

About 30 μL PBS solution was pipetted into the reaction well on the GMR chip that was connected to a GMR biosensing detection system (FIG. 6). An alternating current at 1000 Hz and an alternating in-plane field of 30 Oe at 50 Hz were applied to each GMR sensor. The amplitude of the mixing tone (1050 Hz) is measured as primary output signal by a Fast Fourier Transform of the time-domain voltage signal from a Data Acquisition Card (DAQ, NI USB-6289). A Wheatstone bridge setup shared by all 64 sensors is employed to eliminate the background analog signal, thus the small meaningful signal can be amplified and detected. Each measurement takes about one second, so each GMR sensor can be sampled about once per minute.

After running for about 10 min, about 30 μL of MNPs solution was added, and the detection signal generated by MNPs binding to sensor surface could be recorded in real-time. The MNPs with a size of 50 nm were purchased from Miltenyi Biotech Inc. (catalog no. 130-048-102), and one MNP is composed of several 10 nm iron oxides cores embedded in a dextran matrix. The surfaces of the MNPs are functionalized with streptavidin. These MNPs are dispersed and colloidally stable, so they do not aggregate and settle on sensor surface.

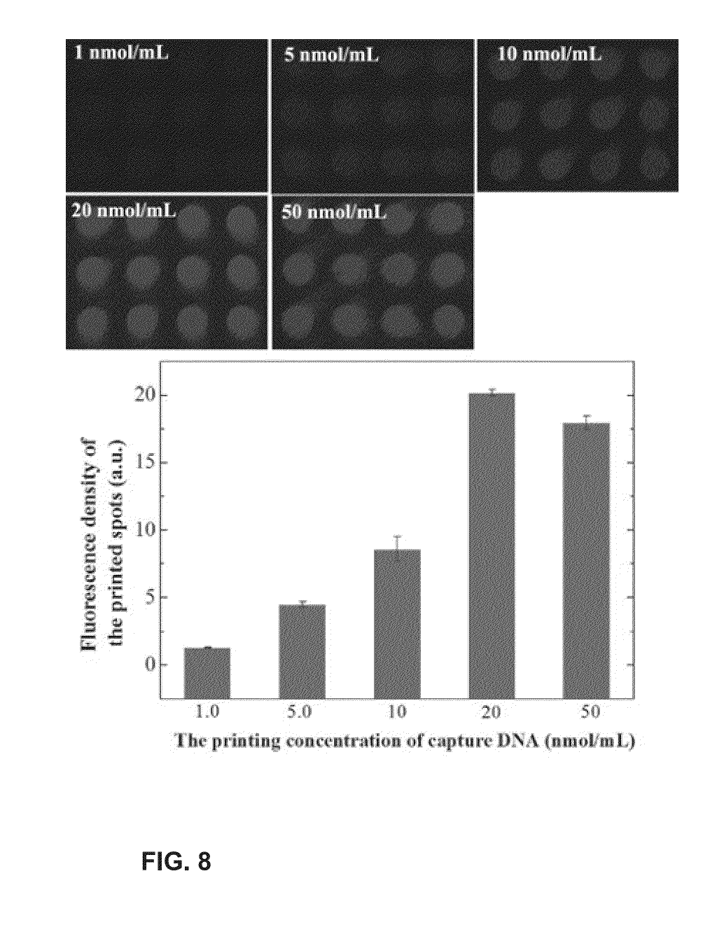

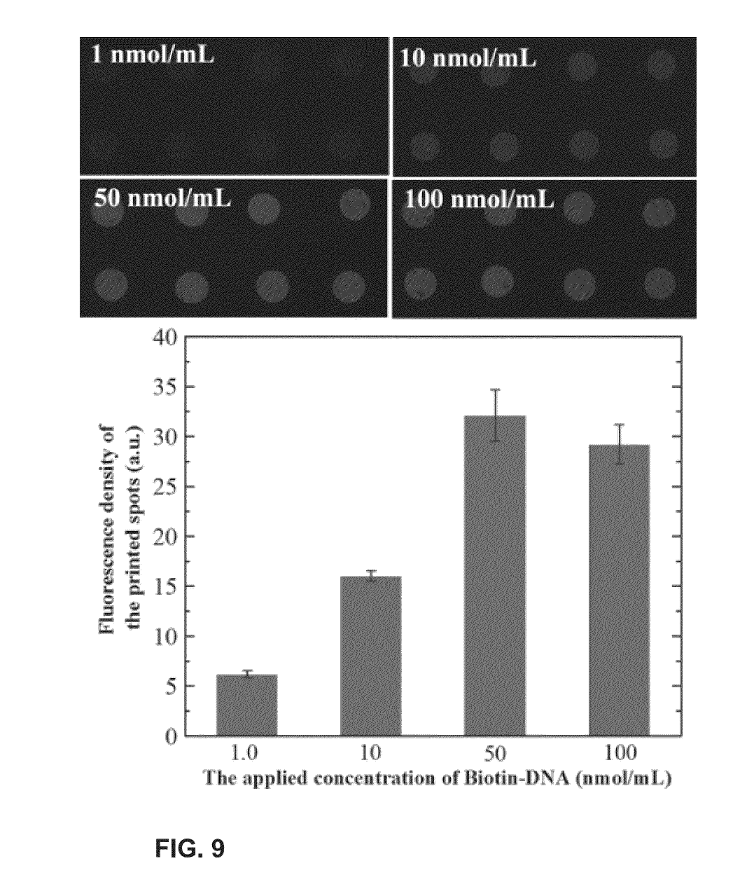

Concentrations of capture DNA and biotin-DNA oligmers were determined before Hg²⁺ assay using GMR biosensors by fluorescence assay. FIGS. 8 and 9 include fluorescence images and bar graphs of fluorescence density versus applied concentration of capture DNA oligomer and biotin-marked DNA, respectively. The capture DNA oligomers (FIG. 8) were printed on APTES and glutaraldehyde modified silicon surface. After incubation, washing and drying, the capture DNA immobilized surfaces were imaged using Olympus IX70 Invert fluorescence microscope under identical camera conditions. Their fluorescence spots and density are shown in FIG. 8. The distance between the centers of adjacent spots is about 400 μm. The capture DNA used here was the same as the capture DNA used for detecting Hg²⁺, aside from being labeled with a fluorescent dye (56-FAM) at the 5′ end.

In a typical DNA sandwich hybridization assay, if more capture DNA is immobilized on the substrate surface, more target DNA would be bound in the same procedural condition. As shown in FIG. 8, the fluorescence intensity of the spots increases as the applied printing concentration goes up from about 1 nmol/mL to about 20 nmol/mL. This indicates that there is an increase in the amount of bound capture DNA. However, the bound amount shows a decrease as the concentration reaches 50 nmol/mL. Thus, the printing concentration of capture DNA was set as about 20 nmol/mL.

As expected, the fluorescence signal generally increases as the Hg²⁺ concentration increases, as shown in FIG. 9. For preparing the samples in FIG. 9, the capture DNA concentration was about 20 nmol/mL; the Hg²⁺ concentration was about 50 μM, and the Streptavidin-AF555 concentration was about 20 μg/mL. The Streptavidin-AF555 was procured from Invitrogen, U.S.A.). After the biotin-DNA was bound, streptavidin-AF555 was added and bound for colorimetric imaging. The fluorescence detection results indicate that the concentration of biotin-DNA also plays an important role in this Hg²⁺ assay. The concentration of biotin-DNA producing the highest fluorescence intensity was found to be 50 nmol/mL. The procedure of this fluorescence assay is similar to that carried out on the GMR sensor surface, except that streptavidin labeled MNPs are replaced by streptavidin-AF555. Thus, the applied concentration of biotin-DNA also uses 50 nmol/mL in the GMR Hg²⁺ assay.

The microarray images in FIGS. 8 and 9 indicate that background signal is very low. FIGS. 8 and 9 demonstrate that the experimental protocol for the biochemical binding part works. Compared to the fluorescence assay, GMR biosensors do not need central laboratory instruments and could potentially realize an in-field analysis. Additionally, GMR biosensors are immune to background interference from environmental water samples, as the detected signal is magnetic rather than light.

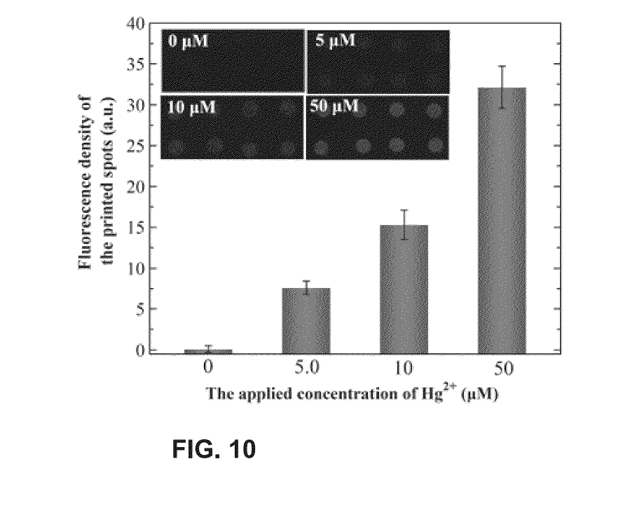

FIG. 10 is an example graph of fluorescence density versus concentration of Hg²⁺. In preparing the data shown in FIG. 10, the capture DNA concentration was about 20 nmol/mL, the biotin-DNA concentration was about 50 nmol/mL, and the Streptavidin-AF555 concentration was about 20 μg/mL. Data is shown as mean plus/minus SD. After the biotin-DNA was bound, streptavidin-AF555 was added and bound for colorimetric imaging. FIG. 10 illustrates that increasing concentration of Hg²⁺ in the sample produces increasing fluorescence, suggesting increasing binding of biotin-DNA.

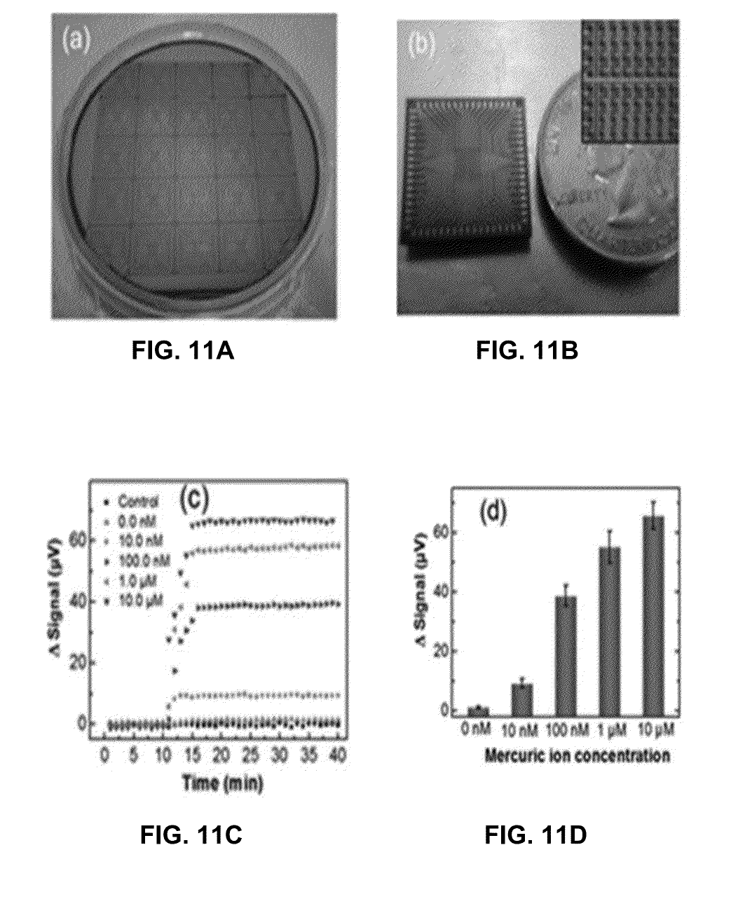

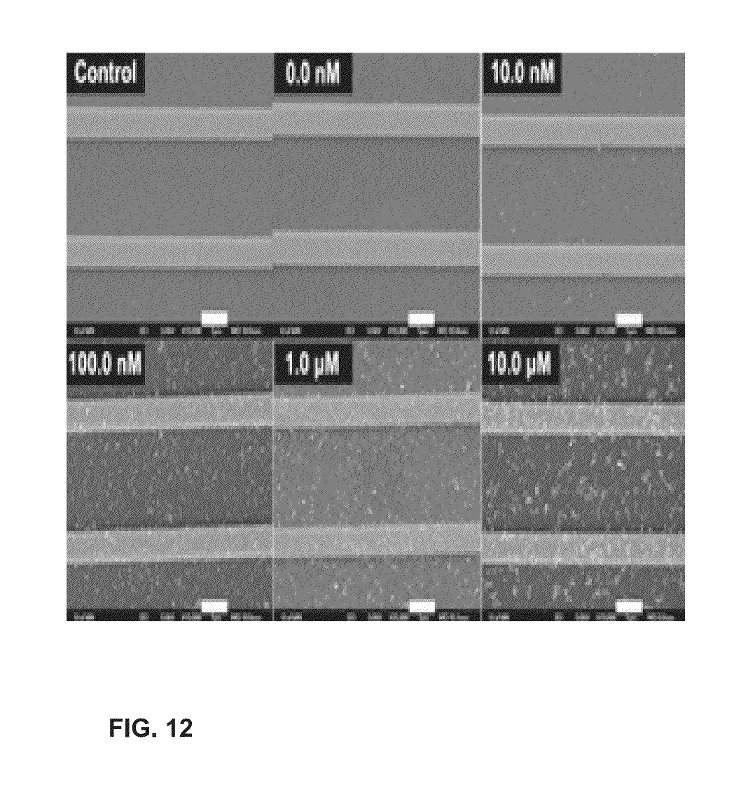

The real-time signals were detected and recorded using a bench-top GMR biosensing system FIG. 6. At present, the system is able to monitor up to 64 sensors in real-time, with a recording rate of 64 data points about every minute. Hence, one data can be recorded for each sensor in one minute. An example real-time binding curves (signal vs. time) for Hg²⁺ assays are shown in FIG. 11(c), and MNPs were added at 10 min. FIG. 11(a) is an image illustrating an example 4-inch silicon wafer, which, in some examples, may produce 21 full GMR biochips and 4 fragmentary GMR biochips. FIG. 11(b) is an image illustrating the size of an example GMR biochip compare to a U.S. quarter. The 64 (8×8 array, inserted image) GMR sensors were located in the central area of the chip, and each sensor was accordingly connected to peripheral contact pads on the periphery of the chip via contact lines. FIG. 11(d) is an example bar diagram illustrating average signals (with standard deviation (SD)) for mercuric ions (Hg²⁺) in buffer. Typically, the signal at time t=30 min is used as the final signal for each sensor. Mean (SD) value of the signals from active sensors on the same GMR biochip are reported to compare different Hg²⁺ assay runs.

No obvious change is observed for the control signal (blank sensor), implying that few MNPs were bound to the control sensor surface. This was verified by SEM analysis, shown in FIG. 12. All the scale bars are 1 μm. After signal measurement was finished, the GMR chip was taken out and washed by water to remove any unbound MNPs immediately, followed by being dried by nitrogen gas. The chip was coated with 5 nm Au film and further investigated by Field Emission Scanning Electron Microscopy (FESEM, JEOL 6500).

The control signal is of great importance in GMR biosensing. It not only indicates whether the testing is stable and repeatable but also indicates the influence of non-specific binding. In absence of Hg²⁺ ([Hg²⁺]=0 nM), the signal is almost negligible compared to the control signal line. The other signals for various Hg²⁺ concentrations show a rise beginning at t=10 min, which the MNPs were added to the sample wells. In this assay, the signal rising reflects real-time MNPs binding to the GMR sensor surface, on which biotin-DNA and Hg²⁺ have already been bound. The signal level for 10 nM Hg²⁺ saturates within about 3 minutes, and reaching equilibrium for Hg²⁺ with higher concentration takes about 5 minutes. More biotin-DNA are bound to the sensor surface as the Hg²⁺ concentration increases. It therefore takes longer time to equilibrate for MNPs binding.

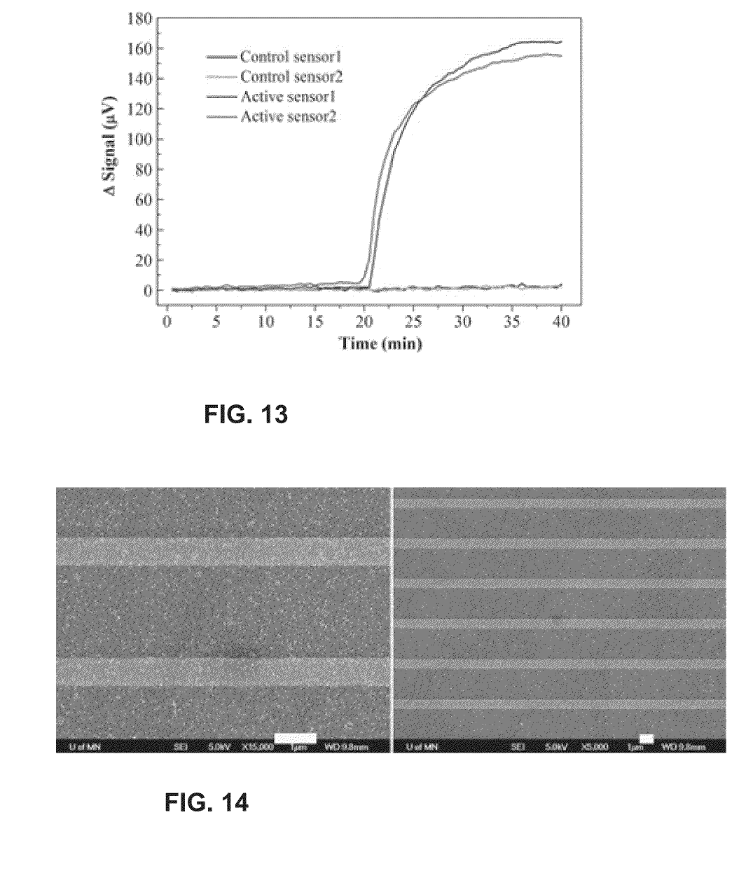

Furthermore, the binding time increases up to about 15 minutes as the saturated signal reaches 150-160 μV, as shown in FIG. 13. BSA (10 mg/mL) and biotinylated antibody (10 μg/mL, catalog no. VJA02, from R&D system) were printed and immobilized on different sensors (on one GMR chip). The signals for these sensors were recorded in real-time. The MNPs (50 nm, Miltenyi Biotech) solution was added at the time of 20 min. The signals for two typical control sensors (covered by BSA) and active sensors (immobilized with biotinylated antibody) are shown here.

FIG. 14 illustrates example SEM images of MNPs binding on biotinylated antibody modified GMR sensor under different magnifications. All the scale bars are 1 μm. The active GMR sensors show a rise in record signal after MNPs are added, reflecting that MNPs are binding to the GMR sensor surface. In contrast, signals for the control GMR sensors do not show obvious change throughout the whole testing process. These binding curves indicate that the GMR sensors work well as expected. The detection signals are exclusively originated from bound MNPs on the GMR sensor surface. Other non-biological factors, such as electronics noise, have not interfered with the output signals. The SEM images in FIG. 14 show that the active GMR sensor surface (left) was densely covered by MNPs, which was consistent with the detection signals.

The average signals for various Hg²⁺ concentrations are shown in FIG. 11(c). The LOD (limit of detection) of this Hg²⁺ assay was about 10 nM (2 μg L⁻¹), which is the maximum contaminant level for mercury in drinkable water regulated by the U.S. Environmental Protection Agency (EPA) in accordance with the authority of the Safe Drinking Water Act.

The magnitude of dynamic range for Hg²⁺ detection using GMR sensing technology is about to 3 orders of magnitude (10 nM to 10 μM). The average signal for 10 nM Hg²⁺ is about 9 μV, and the signal increases with increasing Hg²⁺ concentration. Hg²⁺ detection based on various methods is summarized in Table 1.

TABLE 1

| | Dynamic range | Limit of detection |
|---|---|---|
| Method | (nM) | (nM) |
| Electrochemical sensor | 1.0-500 | 1.0 |
| Triboelectric sensor | 100-5000 | 30 |
| Surface plasmon resonance | 100-2400 | 100 |
| Quartz crystal microbalance | 0.5-100 | 0.24 |
| Quantum dots | 2.5-40 | 2.5 |
| Colloidal gold nanoparticles | 100-2000 | 100 |
| GMR biosensor | 10-10000 | 10 |

Table 1 shows that the proposed GMR biosensor possesses quite wide dynamic range and relative low detection limit for the detection of Hg²⁺ with respect to other potential technologies. The GMR signal responses were further confirmed by SEM analysis of GMR sensor surface. As shown in FIG. 12, the number of bound MNPs on sensor surface obviously increases with increasing Hg²⁺ concentration in the assay. The GMR sensor of the 0 nM Hg²⁺ sample shows very few bound MNPs, while the bound number for 10 μM Hg²⁺ sample is up to about 52/μm².

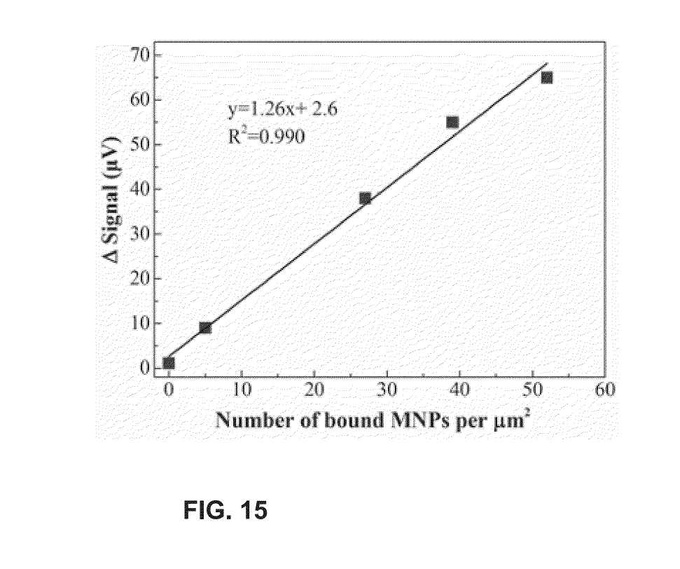

The dependence of the GMR sensor signal on the number of bound MNPs was also analyzed. FIG. 15 is a diagram of the GMR sensor signal versus the number of bound MNPs per μm² on the GMR sensor surface. The data points originated from Hg²⁺ assay with the concentrations ranging from 0 nM to 10 μM. The bound numbers of MNPs were estimated based on SEM results shown in FIG. 12. FIG. 15 includes a linear regression equation fit to the experimental data. The result indicates that the signal versus number of bound MNPs per μm² has a good linear relationship (R²=0.99).

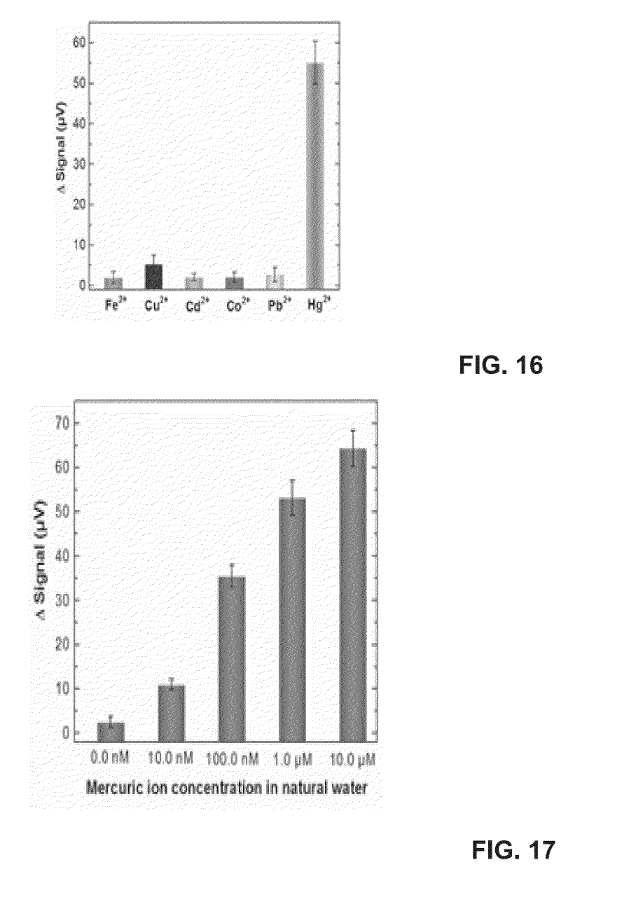

In addition to the sensitivity, this GMR biosensing system also should have a high selectivity towards Hg²⁺ ions. Previous studies have demonstrated that the T-T mismatch is very selective in binding to Hg²⁺ in different DNA-based Hg²⁺ testing systems, and a wide variety of metal ions do not show obvious interference with these methods. To investigate the selectivity of the GMR sensing technique for the detection of Hg²⁺ ions, five common metal ions at a concentration of 1 μM were tested (FIG. 4). FIG. 16 is a bar diagram illustrating the change in signal of a GMR biosensor for each of these six metals. FIG. 16 illustrates that all signal responses of the five metal ions (other than Hg²⁺) are less than 10% of that of Hg²⁺. The responses of the other five metal ions are even weaker than the signal of Hg²⁺ at the limit of detection concentration (10 nM). Thus, this GMR bioassay is highly selective for Hg²⁺ detection.

For the purposes of determining the capability of the GMR bioassay to detect Hg²⁺ in aqueous natural media, Hg²⁺ was spiked in water from Lake Minnetonka in Minnesota. FIG. 17 is a bar diagram illustrating average signals for various Hg²⁺ concentrations in natural water. Data was shown as mean±SD. The original concentration of total mercury in the lake water was determined to be below about 12.5 pM (2.5 ng L⁻¹) by cold vapor atomic fluorescence spectrometry, which is far below the limit of detection of the GMR biosensor assay. As detailed in FIG. 17, the GMR bioassay is able to reliably test Hg²⁺ concentration up to 10 μM, and it also has a limit of detection of about 10 nM for Hg²⁺ in natural water samples. As a new testing method for Hg²⁺, there are multiple feasible strategies to further improve the sensitivity and dynamic range of this prototype GMR biosensing system in future. First, the DNA sequences with designed T-T mismatches could be further developed to bind Hg²⁺ more efficiently. Second, the magnetic performance of GMR sensor may be improved by modifying its shape, composition, and fabrication technology. Finally, MNPs have a significant impact on the GMR sensor signal; thus, choosing MNPs with superior quality (e.g., high-moment magnetic nanoparticles) could greatly improve the sensitivity.

FIGS. 18A-18D are conceptual diagrams that illustrate another example technique by which a magnetic biosensor may detect a concentration of an analyte in a sample. The technique illustrated in FIGS. 18A-18D may be referred to as a three-layer technique or a sandwich technique. As shown in FIGS. 18A-18D, magnetic biosensor 50 may include a magnetic stack 22 and a sample container 24. In the simplified example shown in FIGS. 18A-18D, magnetic stack 22 includes a fixed magnetic layer 26, a nonmagnetic layer 28, and a free magnetic layer 30. In some implementations, magnetic stack 22 may include additional layers. Examples of other magnetic stacks that can be used in magnetic biosensor 20 are described below.

Fixed magnetic layer 26 includes a magnetic material formed in a manner such that a magnetic moment 32 of fixed magnetic layer 26 is substantially fixed in a selected direction under magnetic fields experienced by the fixed magnetic layer 26. As shown in FIGS. 18A-18D, magnetic moment 32 of fixed magnetic layer 26 is fixed in an in-plane direction (i.e., a direction within a major plane of fixed magnetic layer 26). In other examples, magnetic moment 32 may be fixed in a direction out of the plane of fixed magnetic layer 26. For instance, magnetic moment 32 may be fixed at an angle canted out of the plane between about 1 degree and about 90 degrees (where 90 degrees is substantially normal to the major plane of fixed magnetic layer 26). In some implementations, magnetic moment 32 may be fixed in a direction substantially normal (perpendicular) to the major plane of fixed magnetic layer 26. In some examples, magnetic moment 32 of fixed magnetic layer 26 is fixed using one or more additional layers (not shown) in magnetic stack 22, e.g., using anti-ferromagnetic coupling.

Various magnetic materials may be used for forming fixed magnetic layer 26, including, for example, iron-nickel (FeNi), cobalt-iron-boron (CoFeB) alloys, palladium/cobalt (Pd/Co) multilayer structures, combinations thereof, or the like. As another example, a synthesized antiferromagnetic layer (e.g., Co/Ru/Co) could be positioned underneath fixed magnetic layer 26 to fix its magnetization direction. Thickness of fixed magnetic layer 26 may depend on, for example, the material used to formed fixed magnetic layer 26, a thickness of nonmagnetic layer 28, a thickness of free magnetic layer 30, and other variables.

Nonmagnetic layer 28 provides spacing between fixed magnetic layer 26 and free magnetic layer 30. Nonmagnetic layer 28 may include a nonmagnetic material, such as, for example, a non magnetic metal or alloy, or an oxide or a dielectric material. In some examples, nonmagnetic layer 28 may include copper (Cu), silver (Ag), magnesium oxide (MgO), or the like. A thickness of nonmagnetic layer 28 may vary and be selected based upon, for example, properties of free magnetic layer 30 and fixed magnetic layer 26. In an example, nonmagnetic layer 28 may be formed of MgO and have a thickness of about 1.7 nm. In some examples, nonmagnetic layer 28 may be referred to as a spacer layer.

Free magnetic layer 30 includes a magnetic material formed in such a manner to allow a magnetic moment 34 of free magnetic layer 30 to rotate under influence of an external magnetic field (i.e., external to magnetic stack 22). Free magnetic layer 30 is also formed so that magnetic moment 34 of free magnetic layer 30 is oriented in a selected direction in the absence of an external magnetic field (referred to as a magnetically stable state). In the example shown in FIGS. 18A-18D, magnetic moment 34 is formed such that a magnetically stable state is perpendicular to a major plane of free magnetic layer 30. In other examples, magnetic moment 32 may have a magnetically stable state in another direction out of the plane of free magnetic layer 30. For instance, magnetic moment 34 may have a magnetically stable state at an angle canted out of the plane between about 1 degree and about 90 degrees (where 90 degrees is substantially normal to the major plane of free magnetic layer 30). In other implementations, e.g., when magnetic moment 32 of fixed magnetic layer 26 is fixed in a direction out of the plane of fixed magnetic layer 26, magnetic moment 34 of free magnetic layer 30 may have a magnetically stable state parallel to the major plane of free magnetic layer 30.

Free magnetic layer 30 may be formed of magnetic metals or alloys, such as, for example, a FeNi, CoFe, CoFeNi, CoFeN, or CoFeB alloy. A thickness of free magnetic layer 30 may be selected based on a number of variables, including, for example, a selected sensing regime, an external field to be applied to magnetic stack 22, composition and/or thickness of fixed magnetic layer 26 and nonmagnetic layer 28, or the like. In some examples, the thickness of free magnetic layer 30 can be between about 1 nm and about 5 nm, such as about 1.1 nm, about 1.3 nm, about 1.5 nm, about 1.7 nm, or about 2 nm.

Magnetic biosensor 50 also includes a magnetic field generator 46, which may include, for example, a permanent magnetic or an electromagnet. Magnetic field generator 46 generates a substantially constant magnetic field 48 oriented in a direction perpendicular to a major plane of free layer 30. Magnetic field 48 biases magnetic moment 34 of free layer 30 in a direction substantially parallel to magnetic field 48.

Sample container 24 may be formed of any material suitable for containing a sample. For example, Sample container 24 may be formed of a polymer, plastic, or glass that is substantially nonreactive with components of the sample. In some instances, sample container 24 is a reaction well. In other examples, sample container 24 is a microfluidic channel. Sample container 24 may be any suitable shape, including, for example, a hollow cylinder, a hollow cube, an elongated channel, or the like. In some instances, sample container 24 is sized to contain a small amount of sample, e.g., nL or μL of sample. For example, sample container 24 may be sized to contain about 40 μL of sample. In other instances, sample container 24 is sized to contain larger amounts of sample, e.g., mL of sample. For example, sample container 24 can be a cylindrical well with a radius of about 25 millimeters (mm) and a height of about 2 mm, which has a volume of about 3.925 mL.

In some examples, instead of a single sample container 24 being coupled to or associated with a single magnetic stack 22 (as shown in FIGS. 18A-18D), a single sample container 24 may be associated with or coupled to a plurality of magnetic stacks 22. For example, a single sample container 24 may be associated with or coupled to at least four sensors, such as 25 sensors or 64 or 320 sensors.

Within sample container 24 and attached, e.g., chemically bonded, to a surface of sample container 24 are a plurality of capture molecules 44 (e.g., capture DNA oligomers). Capture DNA oligomers 44 may be selected to capture molecules of interest in the sample disposed within sample container 24, such as Hg²⁺ ions. Although a single type of capture DNA oligomers 44 is shown in FIGS. 18A-18D, in other examples, multiple types of capture DNA oligomers 44 (e.g., configured to capture different molecules of interest) may be attached to the surface of sample container 24, e.g., at different locations of sample container 24. In some implementations, when sample well 24 includes a plurality of different types of capture DNA oligomers 44, each type of capture DNA oligomers 44 may be disposed adjacent to a different magnetic stack 22. For example, a single type of capture DNA oligomers 44 may be associated with a single magnetic stack 22, and a sample container 24 may be associated with a plurality of magnetic stacks 22.

In the three-layer technique, a sample including analyte 52 (e.g., water that may or may not include Hg²⁺ ions) and biotin labeled DNA 54 is first deposited in sample container 24, as shown in FIG. 18A. The biotin labeled DNA 54 may include T-T mismatches relative to the capture molecules 44 (e.g., capture DNA oligomers). In the absence of Hg2+, biotin labeled DNA 54 is rarely hybridized to capture DNA oligomers 44 because of the mismatched base pairs. In contrast, the biotin labeled DNA 54 can be bound and hybridized to the capture DNA oligomers 44 attached to the GMR sensor surface in the presence of Hg²⁺ due to the T-Hg²⁺-T complex and Watson-Crick base pairing. Analyte 52 is allowed time to bind to capture DNA oligomers 44 and biotin labeled DNA 54 is allowed to time to bind to analyte 52 and capture DNA oligomers 44, as shown in FIG. 18B. Once analyte 52 and biotin labeled DNA 54 have been allowed time to bind to capture DNA oligomers 44, the sample is removed and, in some implementations, the sample container may be rinsed with a solvent to remove any sample residue.

As shown in FIG. 18C, a solution containing MNPs 40, is introduced into the sample chamber 24. MNPs 40 may be functionalized with streptavidin, which interacts with biotin to bond the MNPs 40 to biotin labeled DNA 54. MNPs 40 can include a high magnetic moment material such as FeCo, FeCoN. FeSi, FeC, FeN, combinations of Fe, N, C, Si, or the like. MNPs 40 can be fabricated using various techniques, including physical vapor nanoparticle-deposition. The size of MNPs 40 can be controlled to be in the range of, for example, 3 to 100 nm, such as about 20 nm or about 50 nm. Because the size and shape of MNPs 40 affect the magnetic properties of MNPs 40, which affects operation of magnetic stack 22, the size and shape of MNPs 40 may be controlled to be substantially uniform. In some examples, MNPs 40 may be substantially cubic in shape and substantially the same size, e.g., defined by a width of the respective one of MNPs 40.

As shown in FIG. 18D, MNPs 40 bond to biotin labeled DNA 54. After sufficient time to allow bonding, the solution and excess MNPs 40 may be removed and a voltage applied across magnetic stack 22 to measure the resistance of magnetic stack 22. As described above, the resistance of magnetic stack is a function of the relative orientations of magnetic moment 32 of fixed layer 26 and magnetic moment 34 of free layer 30. As the orientation of magnetic moment 34 is affected by the magnetic fields generated by MNPs 40, the resistance may be change based on the number of biotin labeled DNA 54 bound to analytes 52. For example, as shown in FIG. 18D, the magnetic fields generated by MNPs 40 affect magnetic moment 34 in a downward direction of FIG. 18D. This change of magnetic moment 34 of free layer 30 changes a magnetoresistance of magnetic stack 22, which may be measured by sending applying a voltage across magnetic stack 22 and measuring the resulting current. After generating a calibration curve of measured current versus known concentration of analyte 52, the calibration curve and measured current across magnetic stack 22 may be used to determine a concentration of analyte in new samples.

FIGS. 19A and 19B are conceptual diagrams that illustrate another technique by which a magnetic biosensor 70 may detect a concentration of an analyte in a sample. The technique illustrated conceptually in FIGS. 19A and 19B may be referred to as detection by competition. As shown in FIGS. 19A and 2B, magnetic biosensor 70 may include a magnetic stack 22 and a sample container 24. In the simplified example shown in FIGS. 19A and 19B, magnetic stack 22 includes a fixed magnetic layer 26, a nonmagnetic layer 28, and a free magnetic layer 30. In some implementations, magnetic stack 22 may include additional layers. Examples of other magnetic stacks that can be used in magnetic biosensor 70 are described above with respect to FIGS. 18A-18D.

Magnetic biosensor 70 also includes a magnetic field generator 46, which may include, for example, a permanent magnetic or an electromagnet. Magnetic field generator 46 generates a substantially constant magnetic field 48 oriented in a direction perpendicular to a major plane of free layer 30. Magnetic field 48 biases magnetic moment 34 of free layer 30 in a direction substantially parallel to magnetic field 48.

Sample container 24 may be formed of any material suitable for containing a sample, including a polymer, plastic, or glass that is substantially nonreactive with components of the sample. Further details of sample container 24 are described above with respect to FIGS. 18A-18D.

Within sample container 24 and attached, e.g., chemically bonded, to a surface of sample container 24 are a plurality of capture molecules or capture antibodies 44. Capture antibodies 44 may be selected to capture molecules of interest in the sample disposed within sample container 24. Although a single type of capture antibodies 44 is shown in FIGS. 19A and 19B, in other examples, multiple types of capture antibodies 44 (e.g., configured to capture different molecules of interest) may be attached to the surface of sample container 24, e.g., at different locations of sample container 24. In some implementations, when sample well 24 includes a plurality of different types of capture antibodies 44, each type of capture antibodies 44 may be disposed adjacent to a different magnetic stack 22. For example, a single type of capture antibodies 44 may be associated with a single magnetic stack 22, and a sample container 24 may be associated with a plurality of magnetic stacks 22.

In a detection-by-competition technique, a sample, which includes a plurality of unmarked analytes or unmarked antigens 78, and a reagent, which includes a plurality of magnetically marked analytes 72, are mixed and deposited in sample container 24, as shown in FIG. 19A. Magnetically marked analytes 72 include a molecule of interest, also referred to as a magnetically marked antigen 74. Magnetically marked antigen 74 may be the same molecule as unmarked antigens 78 or may possess the same binding properties (to capture antibodies 44) as unmarked antigens 78.

Magnetically marked antigen 74 is bound to a magnetic nanoparticle (MNP) 76. MNPs 76 can include a high magnetic moment material such as FeCo, FeCoN, FeSi, FeC, FeN, combinations of Fe, N, C, Si, or the like. MNPs 76 can be fabricated using various techniques, including physical vapor nanoparticle-deposition. The size of MNPs 76 can be controlled to be in the range of, for example, 3 to 100 nm. Because the size and shape of MNPs 76 affect the magnetic properties of MNPs 76, which affects operation of magnetic stack 22, the size and shape of MNPs 76 may be controlled to be substantially uniform. In some examples, MNPs 76 may be substantially cubic in shape and substantially the same size, e.g., defined by a width of the respective one of MNPs 76.

As shown in FIG. 19B, magnetically marked antigens 72 and unmarked antigens 78 compete to bind at capture antibodies 44. Because of this, the number of magnetically marked antigens 72 bound by capture antibodies 44 is inversely proportional to the concentration of unmarked antigens 78 in the sample. The MNPs 76 generate magnetic fields, which affect the orientation of magnetic moment 34. For example, as shown in FIG. 19B, the magnetic fields generated by MNPs 76 of magnetically marked analytes 72 captured by capture antibodies 44 affects magnetic moment 34 in a downward direction of FIG. 19B. This change of magnetic moment 34 of free layer 30 changes a magnetoresistance of magnetic stack 22, which may be measured by sending applying a voltage across magnetic stack 22 and measuring the resulting current. After generating a calibration curve of measured current versus known concentration of unmarked antigens 78, the calibration curve and measured current across magnetic stack 22 may be used to determine a concentration of unmarked antigens 78 in the sample.

Although the preceding examples have been described with respect to a GMR magnetic sensor, in other examples, the magnetic biosensing system described herein may be used with different types of magnetic sensors, such as magnetic tunnel junction (MTJ) sensors that may have the spin valve structure, Hall sensors that may have the spin valve structure, giant magnetoimpedence (GMI) sensors, or the like. Similarly, although the preceding examples have been described with respect to the detection of mercuric ions, in other examples, other metal ions could be detected using different binding and chemical systems with the sensors described herein.

In this work, a highly sensitive, selective and real-time Hg²⁺ detection method using a GMR biosensing scheme combined with T-Hg²⁺-T coordination chemistry was developed. A limit of detection of 10 nM in both buffer and natural water, which is the maximum mercury level in drinking water defined by US EPA, was achieved. Three orders of detection dynamic range (10 nM to 10 μM) in the GMR Hg²⁺ bioassay were obtained. Based on the features of GMR biosensing technology, this GMR Hg²⁺ bioassay suggests a convenient and rapid field test. Furthermore, as a versatile and strong contender in molecular diagnostics, GMR bioassay not only can be applied in Hg²⁺ detection, but also has great potential for the application of other pollutant monitoring in environment and food samples.

Various examples have been described. These and other examples are within the scope of the following claims.

1: A system comprising:
a magnetic sensor comprising a free magnetic layer and a fixed magnetic layer;
a sample container disposed over the magnetic stack;
a plurality of capture DNA oligomers on a surface of the magnetic biosensor within the volume defined by the sample container above the magnetic stack, wherein each of the plurality of capture DNA oligomers includes at least one thymine base configured to bind to an Hg²⁺ ion;
a magnetic field generator configured to generate a magnetic field that influences the free layer; and
circuitry configured to measure a resistance of the magnetic sensor.

2: The system of claim 1, wherein the magnetic sensor comprises a plurality of magnetic sensors formed in a single substrate.

3: The system of claim 1, wherein the resistance of the magnetic sensor is proportional to a concentration of Hg²⁺ ions in a sample disposed in the sample container.

4: The system of claim 1, wherein the free magnetic layer comprises a magnetic moment oriented in a plane of the magnetic sensor in a stable state, and wherein the fixed magnetic layer comprises a magnetic moment oriented in a plane of the magnetic sensor in a stable state.

5: The system of claim 1, wherein at least one of the free magnetic layer comprises a magnetic moment oriented out of a plane of the magnetic sensor in a stable state.

6: The system of claim 1, wherein the plurality of capture DNA oligomers comprise 5′/ACTAACTACTGTATCCTGCA/3AmMC6T/3′ (SEQ ID NO: 1) with amino modification at the 3′ end.

7: A magnetic biosensor array comprising:
a plurality of electrical contacts located along at least one peripheral edge of the magnetic biosensor array;
a sample container;
a plurality of the magnetic biosensors each located adjacent to a surface of the magnetic biosensor array and comprising a magnetic sensor comprising a free magnetic layer and a fixed magnetic layer, and
a plurality of capture DNA oligomers on a surface of the magnetic biosensor within the volume defined by the sample container above the plurality of the magnetic biosensors, wherein each of the plurality of capture DNA oligomers includes at least one thymine base configured to bind to an Hg²⁺ ion.

8: The magnetic biosensor array of claim 7, further comprising:
a magnetic field generator configured to generate a magnetic field that influences the respective free layer of each of the plurality of the magnetic biosensors; and
circuitry configured to measure a respective resistance of each of the plurality of the magnetic biosensors.

9: The magnetic biosensor array of claim 7, wherein the resistance of the magnetic sensor is proportional to a concentration of Hg²⁺ ions in a sample disposed in the sample container.

10: The magnetic biosensor array of claim 7, wherein the free magnetic layer comprises a magnetic moment oriented in a plane of the magnetic sensor in a stable state, and wherein the fixed magnetic layer comprises a magnetic moment oriented in a plane of the magnetic sensor in a stable state.

11: The magnetic biosensor array of claim 7, wherein at least one of the free magnetic layer comprises a magnetic moment oriented out of a plane of the magnetic sensor in a stable state.

12: The magnetic biosensor array of claim 7, wherein the plurality of capture DNA oligomers comprise 5′/ACTAACTACTGTATCCTGCA/3AmMC6T/3′ (SEQ ID NO: 1) with amino modification at the 3′ end.

13: A kit comprising:
a magnetic biosensor comprising:
a magnetic sensor comprising a free magnetic layer and a fixed magnetic layer;
a sample container disposed over the magnetic stack; and
a plurality of capture DNA oligomers on a surface of the magnetic biosensor within the volume defined by the sample container above the magnetic stack;
a solution including a plurality of biotin-labeled DNA;
a solution including a plurality of streptavidin labeled magnetic nanoparticles; and
instructions for introducing a sample into the sample container, introducing the solution including the plurality of biotin-labeled DNA into the sample container, and introducing the solution including the plurality of streptavidin labeled magnetic nanoparticles into the sample container to detect a concentration of Hg²⁺ ions in the sample.

14: The kit of claim 13, wherein the magnetic biosensor further comprises:
a magnetic field generator configured to generate a magnetic field that influences the free layer; and
circuitry configured to measure a resistance of the magnetic sensor.

15: The kit of claim 13, wherein the resistance of the magnetic sensor is proportional to a concentration of Hg²⁺ ions in a sample disposed in the sample container.

16: The kit of claim 13, wherein the free magnetic layer comprises a magnetic moment oriented in a plane of the magnetic sensor in a stable state, and wherein the fixed magnetic layer comprises a magnetic moment oriented in a plane of the magnetic sensor in a stable state.

17: The kit of claim 13, wherein at least one of the free magnetic layer comprises a magnetic moment oriented out of a plane of the magnetic sensor in a stable state.

18: The kit of claim 13, wherein the plurality of capture DNA oligomers comprise 5′/ACTAACTACTGTATCCTGCA/3AmMC6T/3′ (SEQ ID NO: 1) with amino modification at the 3′ end.

19: The kit of claim 13, wherein the Biotin-labeled DNA comprises 5′/TGCTGGTTTCTGTTGTTTGT/BiotinBB-/3′ (SEQ ID NO: 2).

20: The kit of claim 13, wherein the streptavidin labeled magnetic nanoparticles comprise iron oxides cores embedded in a dextran matrix, functionalized with streptavidin.

21: A method for forming a magnetic biosensor, the method comprising:
forming a magnetic sensor comprising a free magnetic layer and a fixed magnetic layer, wherein at least one of the free layer or the fixed layer has a magnetic moment oriented out of a major plane of the free layer or the fixed layer, respectively, in an absence of an external magnetic field;
placing a sample container over the magnetic stack; and
attaching a plurality of capture DNA oligomers to a surface of the magnetic sensor, wherein each of the plurality of capture DNA oligomers includes at least one thymine base configured to bind to an Hg²⁺ ion.

22: The method of claim 21, wherein the plurality of capture DNA oligomers comprise 5′/ACTAACTACTGTATCCTGCA/3AmMC6T/3′ (SEQ ID NO: 1) with amino modification at the 3′ end.

23: The method of claim 21, wherein the free magnetic layer comprises a magnetic moment oriented in a plane of the magnetic sensor in a stable state, and wherein the fixed magnetic layer comprises a magnetic moment oriented in a plane of the magnetic sensor in a stable state.

24: The method of claim 21, wherein at least one of the free magnetic layer comprises a magnetic moment oriented out of a plane of the magnetic sensor in a stable state.

25: A method for detecting a concentration of Hg²⁺ ions in a sample, the method comprising:
introducing a sample including Hg²⁺ ions into a sample container, wherein the sample container defines a volume adjacent to a magnetic biosensor comprising a magnetic sensor comprising a free magnetic layer and a fixed magnetic layer, a sample container disposed over the magnetic stack, and a plurality of capture DNA oligomers on a surface of the magnetic biosensor within the volume defined by the sample container above the magnetic stack;
introducing a solution including a plurality of biotin-labeled DNA into the sample container;
introducing a solution including a plurality of streptavidin labeled magnetic nanoparticles into the sample container, and
detecting a resistance of the magnetic sensor.

26: The method of claim 25, wherein further comprising mixing the sample including Hg²⁺ ions and the solution including the plurality of biotin-labeled DNA, and wherein the sample including Hg²⁺ ions and the solution including the plurality of biotin-labeled DNA are introduced into the sample container as a mixture.

27: The method of claim 25, wherein the resistance of the magnetic sensor is proportional to a concentration of Hg²⁺ ions in a sample disposed in the sample container.

28: The method of claim 25, wherein the free magnetic layer comprises a magnetic moment oriented in a plane of the magnetic sensor in a stable state, and wherein the fixed magnetic layer comprises a magnetic moment oriented in a plane of the magnetic sensor in a stable state.

29: The method of claim 25, wherein at least one of the free magnetic layer comprises a magnetic moment oriented out of a plane of the magnetic sensor in a stable state.

30: The method of claim 25, wherein the plurality of capture DNA oligomers comprise 5′/ACTAACTACTGTATCCTGCA/3AmMC6T/3′ (SEQ ID NO: 1) with amino modification at the 3′ end.

31: The method of claim 25, wherein the Biotin-labeled DNA comprises 5′/TGCTGGTTTCTGTTGTTTGT/BiotinBB-/3′ (SEQ ID NO: 2).

32: The method of claim 25, wherein the streptavidin labeled magnetic nanoparticles comprise iron oxides cores embedded in a dextran matrix, functionalized with streptavidin.
